(19) United States
(12) Patent Application Publication
(10) Pub. No.: US 2020/0030138 A1
(43) Pub. Date: Jan. 30, 2020

(54) Compartmentalized Heat Transfer Wrap and Method

(71) Applicant: Nikola M. Samson, Las Vegas, NV (US)

(72) Inventor: Nikola M. Samson, Las Vegas, NV (US)

(21) Appl. No.: 16/048,189

(22) Filed: Jul. 27, 2018

(51) Int. Cl. A61F 7/02; A47G 9/02

(52) U.S. Cl. CPC A61F 7/02; A47G 9/0215; A61F 2007/0021; A61F 2007/0268; A61F 2007/0215

(57) ABSTRACT

A compaitmentalized heat transfer wrap for absorbing heat from a living body including a moisture-proof, outer envelope being sealed about a periphery thereof to form an outer boundary, the outer envelope being compartmentalized into a plurality of individual sealed pockets wherein each of the sealed pockets is isolated from each of the remaining individual sealed pockets, each of the individual sealed pockets including a formulated cooling medium encapsulated therein, the cooling medium having a combination of components including carboxymethyl cellulose in powder form, water, and a stabilizing additive for absorbing heat generated by a living body in contact with the outer envelope while the cooling medium is employed and functional at ambient temperature.

This patent application is being flied as a non-provisional patent application under 35 USC Section 111(a).

### BACKGROUND OF THE INVENTION

### Technical Field

The present invention relates to heat transfer devices for absorbing heat from a living body. More specifically, the present invention relates to methods and apparatus fOr a compartmentalized heat transfer wrap and method therefore having a moisture proof, geometrically-shaped, outer envelope that incorporates therein a plurality of individual sealed pockets each including a formulated cooling medium encapsulated therein for absorbing heat generated by a living body in contact with the outer envelope while the cooling medium is employed and functional at ambient temperature.

### Background Art

A plurality of cooling products have been known in the past for use in drawing heat from any number of surfaces including the surface areas of the human body. Heat transfer products known in the prior art include many types of containers including ice packs, frozen gel products, and mechanical heat transfer devices. The ice pack and frozen gel products can be obtained in most any pharmacy or medical supply outlet and can be placed in a freezer portion of any residential refrigerator, After being frozen, the ice pack or frozen gel product can be applied directly to the surface area of the human body to draw heat from the surface to be cooled. Although generally an inexpensive solution and suitable for home usage problems associated with this type of cooling apparatus include that they must constantly be re-frozen and towel wrapped prior to use to avoid moisture leakage and freezer burns to the human surface area being coded.

Mechanical heat transfer devices mentioned immediately above can be fashioned in the form of a blanket-type article and can include a prime mover mechanism that pumps heat absorbing fluids or gels through a miniatures piping system formed in any type of blanket-type article that can be applied to a surface being cooled including surface areas of the human body. The heat collected by the heat absorbing fluid or gel is then transferred to the ambient air or to a secondary fluid or gel through an associated heat exchanger. This type of device, which would normally be found in a hospital or emergency treatment center, has the drawbacks of being large and balky, very expensive and not practical for use in a residential setting would be convenient and an advance in the art if a heat transfer apparatus was available for direct application to surfaces of the human body for dissipating heat therefrom and reducing inflammation in soft tissue, was fashioned in the form of a simple blanket-type article that was easy to utilize and store, that employed a heat, absorbing medium that functioned to absorb heat at ambient temperature and did not require constant refreezing prior to use, and was economical to use and avoided the high cost of more sophisticated mechanical heat transfer devices.

Prior art patents and publications directed to conventional heat transfer devices for absorbing heat from a living body will now be mentioned that may be relevant to the enclosed compartmentalized heat transfer wrap of the present invention.

Prior art patents and publications directed to heat transfer devices will now be mentioned that may be relevant to the enclosed compartmentalized heat transfer wrap of the present invention.

In U.S. Pat. No. 6,523,354 issued to Tolbert on Feb. 25, 2003, entitled Cooling Blanket, there is disclosed a cooling blanket having multiple pockets to contain a heat, sink with the heat sink provided in the pocket. The heat sink can be either a thermoelectric cooling unit or a cold pack. A schematic diagram shown in FIG. 3 illustrates a thermoelectric cooling unit 12 and a control unit 11 having both an AC power source and an alternative DC battery source. FIG. 1 shows a perspective view of the circuit arrangement of the cooling blanket. In column 4 beginning with line 5, FIG. 2 is described as reciting heat sink 12 as a thermoelectric cooling unit, or in the alternative, thermal gel heat sink 28 is described as a cold pack.

In U.S. Patent Application Publication No, 2018/0098879 published by Smith et al, on Apr. 12, 2018 and entitled Thermoregulating Gelpack, there is disclosed a gel-pack having a plurality of self-contained components organized as layers in an interior chamber of the gel-pack. Each layer may be inserted into, and removed from, the interior chamber individually. The components include a solid-state gel layer that stores thermal energy, a first radiant energy barrier that prevents the transfer of thermal energy radiating from an exterior of the gel-pack into the solid-state gel layer, and a second radiant energy barrier that reflects the thermal energy radiating from the user's body into the solid-state gel layer. In paragraph [0046] on page 4 of the Detailed Description of the 2018/0098879 publication, Smith et al. discloses that according to the present invention, a user may remove one or more of the layers, individually, and place them into a freezer or refrigerator or any device capable of cooling the materials that comprise these layers.

In U.S. Pat. No. 5,072,455 issued to St. Ours on Dec. 17, 1991 entitled Heat-Intercepting Garment or Blanket, there is disclosed a heat-intercepting shield or shroud in the form of a garment, blanket or belt having on at least one surface thereof one or more pockets to receive replaceable packets of refreezable material which absorb large quantities of heat as the material melts. Each pocket has two coextensive fabric layers stitched together to form opposite pocket walls. Over the entire pocket area and next to the one wall are coextensive layers of no breathable continuous reflective material and of insulating material. These materials are flexible and are stitched next to that one pocket wall in seams at the edge of the pocket so that both heat reflection and retardation of heat flow by the insulation occurs over the entire pocket area. Between the layer of insulation and the other pocket wall is a space for a heat absorbing packet or packets of frozen gel. The side of the shield or shroud with the packet or packets is placed toward the object to be cooled or protected from external radiant, conductive or convective heat. At column 6 beginning at line 12, the St Ours specification recites that each of the compartments of packet 60 contains a refreezable heat dissipating means in the form of a pack 63 of refreezable non-toxic chemical refrigerant gel.

In U.S. Patent Publication No. 2006/0276089 published on Dec. 7, 2006 by Amarasinghe et al., an article of clothing is described as comprised of having a Phase Change Material (PCM) which is a blend of at least two compounds and which has a melting point of from 5 degrees Centigrade-to-30 degrees Centigrade, and a melting temperature range of from 1 degree Centigrade-to-5 degrees Centigrade, in paragraph [0022] on page 2 Amarasinghe et al. discloses that given the melting point: of this Phase Change Material (PCM), a freezer is usually required in order to “activate” it prior to use by storage in an air conditioned room (below about 18 degrees Centigrade), in a refrigerator or in a freezer.

In U.S. Patent Application Publication No. 2010/0186435 published on Jul. 29, 2010 by Vogel et al., an assembly is disclosed for cooling the body of a person or another body which has cooling elements that are movably connected to one another, contain a cooling liquid, and are arranged in the form of a cape or shawl which can be worn about the body of the person or the like for the purposes of being cooled. In order to provide such a cooling arrangement, which allows particularly rapid and efficient cooling of the body, provision is made for the cooling elements to contain within them in a material, for example graphite, which is a good thermal conductor in relation to the cooling liquid and is intended for absorbing the cooling liquid. In paragraph [0008] on page 1, right column, Vogel et al. discloses that the cape is stored, e.g. in a cooling device such as, e.g. deep-freezer, in order to appropriately cool down the cooling liquid present in the cooling elements. Before the cape is used, it is taken from the deep-freezer and the individual or the like puts it on. In this manner, a sportsman may, for example, cool down the body by wearing the cape for a certain time, e.g. for a few minutes, during the break of matches and may increase his/her performance for the following sports activities.

Therefore, there is a need in the art for a heat transfer apparatus for absorbing heat from a living body, either human or animal, that includes a moisture-proof, outer envelope sealed at the periphery thereof for providing an outer boundary, wherein the outer envelope is compartmentalized into a plurality of individual quilted sealed pockets, each pocket isolated from all remaining sealed pockets and including a formulated cooling medium therein, for absorbing heat generated by a living body in contact with the outer envelope while the cooling medium is employed and functional at ambient temperature and is not required to be refrigerated or frozen prior to use to effect heat transfer from the living body to the formulated cooling medium encapsulated in the individual pockets.

### DISCLOSURE OF THE INVENTION

Briefly, and in general terms, the present invention provides a new and improved compartmentalized heat transfer wrap and method for absorbing heat from a living body. The heat transfer wrap can be applied directly to the surface of the human body for dissipating heat therefrom thereby reducing associated fever and inflammation in soft tissue, surgical incision sites, and problems associated with female menopause, breast feeding/nursing babies, mastitis, and lower back lumbar pain problems.

The heat transfer wrap of the present invention can be provided in the form of a blanket-like article such that the user can lay upon or drape over an effected area of the user's body for absorbing heat therefrom. The invention is ideal for use with toddlers and adults alike and can be utilized to address each of the problems enumerated immediately above. Further, the inventive heat transfer wrap is intended for use at ambient temperature which has been shown during the development and testing phase to be sufficient to lower the body temperature, dissipate heat and reduce inflammation in the user. This significant feature of the present invention enables the heat transfer wrap to be utilized as intended without the requirement of freezing or chilling the wrap prior to use.

The present invention is embodied in a compartmentalized heat transfer wrap for absorbing heat from a living body, either human or animal, that includes a moisture proof, generally geometrically-shaped, outer envelope which is sealed about the periphery thereof to form an outer boundary. The sealing means is dependent upon the material used to form the outer envelope. In the present invention, the prototype design utilizes nylon to form the outer envelope due to nylon being moisture-proof and having heat transfer characteristics. However, other materials can be used such as, for example, robust flexible plastic. Consequently, the sealing of the outer envelope to form the outer boundary can be a pressure heat seal or other suitable sealing means. The material must be of the type that enables the transfer of heat there through and typically is formed in a geometric shape such as a rectangle, square, circle or other desirable shape. The geometric-shaped wrap is intended to have a center of gravity that improves the weight distribution and consequently the handling characteristics thereof.

The outer envelope of the heat transfer wrap is compartmentalized into a plurality of individual sealed pockets. Each of the individual sealed pockets is isolated from each of the remaining sealed pockets, typically by suitable pressure heat seals. The number of individual sealed pockets in the present heat transfer wrap as illustrated in the accompanying drawing Figs. is eight and are distributed in a rectangular arrangement. This number of sealed pockets has been determined to provide a suitable sized blanket-like article and a conveniently positioned center of gravity to improve handling characteristics. However, other arrangements of sealed pockets in other geometric patterns may also be effective. Notwithstanding the geometric shape of the heat transfer wrap and the number of individual quilted pockets formed therein, the outer envelope may be conveniently folded along each of the pressure heat seals for convenient storage.

Each of the individual sealed pockets includes a specially formulated cooling medium encapsulated therein for absorbing heat generated by the living body that is in contact with the outer envelope of the heat transfer wrap. Thus, the function of the formulated cooling medium is to absorb and dissipate the heat generated by the living body. The specially formulated cooling medium includes a combination of components blended to absorb heat including carboxymethyl cellulose in powder form, water, and a stabilizing additive. This combination of components physically exists as a cooling gel and is designed to functionally absorb heat when employed at ambient temperature, e.g., the cooling gel is not required to be frozen or cooled prior to use. This design feature provides a significant advance over the cooling devices known in the prior art.

An optional feature of the present invention includes a flat sleeve enclosure for surrounding the outer envelope of the heat transfer wrap. The flat sleeve enclosure surrounds the outer envelope similar to how a mattress cover surrounds a sleeping mattress. A closure means such as a hook and loop fastener enables the flat sleeve enclosure to totally enclose the outer envelope. The outer envelope as presented herein is fashioned from nylon but the flat sleeve enclosure can provide a sanitary comfortable surface comprised of a cotton or fleece-like material which may be laundered regularly when used with small children and infants. Further, the flat sleeve enclosure can include esthetics or designs imprinted thereon that are suitable for young children.

The present invention is generally directed to a compartmentalized heat transfer wrap for absorbing heat from a living body including a moisture-proof, outer envelope being sealed about a periphery thereof to form an outer boundary. The outer envelope is compartmentalized into a plurality of individual sealed pockets wherein each of the sealed pockets is isolated from each of the remaining individual sealed pockets. Further, each of the individual sealed pockets includes a formulated cooling medium encapsulated therein, the cooling medium having a combination of components including carboxymethyl cellulose in powder form, water, and a stabilizing additive for absorbing heat generated by a living body in contact with the outer envelope while the cooling medium is employed and functional at ambient temperature.

These and other objects and advantages of the present invention will become apparent from the following more detailed description, taken in conjunction with the accompanying drawings which illustrate the invention, by way of example.

### BRIEF DESCRIPTION OF THE DRAWINGS

FIG. 1 is a top plan view of a compartmentalized heat transfer wrap and method of the present invention showing multiple nylon covered quilted pockets with each pocket incorporating a special formulated cooling gel and separated by pressure heat seals;

FIG. 2 is a top plan view of a single nylon covered quilted pocket containing the formulated cooling gel and showing pressure heat seals for separating adjacent quilted pockets and for sealing an outer boundary, and further showing the cross-sectional line 4-4 for referring to FIG. 4;

FIG. 3 is a top plan view of four adjacent quilted pockets intersecting at a common point on the surface of the compartmentalized heat transfer wrap, the quilted pockets being separated by heat seals and each containing the formulated cooling gel;

FIG. 4 is a cross-sectional view of the compartmentalized heat transfer wrap of the present invention taken along the line 4-4 of FIG. 2 showing the interior of one of the plurality of quilted pockets and the formulated cooling gel therein;

FIG. 5 is a planar illustration of the center of gravity of the geometric arrangement of the plurality of equally-weighted quilted pockets of the compartmentalized heat transfer wrap of FIG. 1; and

FIG. 6 is a perspective view of an optional sleeve for enclosing the compartmentalized heat transfer wrap therein for providing a softer, washable, outer surface more compatible for use with an infant utilizing the present invention and showing an exemplary hook and loop closing means.

### DETAILED DESCRIPTION OF THE INVENTION

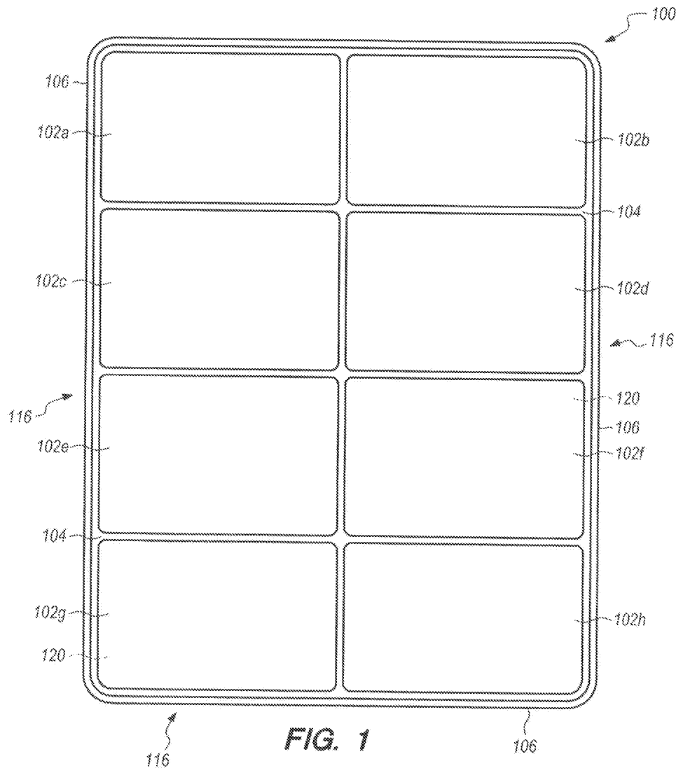

The present invention relates to a compartmentalized heat transfer wrap and method 100 as shown in FIGS. 1-5 (hereinafter referred to as the heat transfer wrap 100). The heat transfer wrap 100 is intended for use in facilitating the absorption of heat from a living body, either human or animal. In the description that follows, it will be assumed that the heat transfer wrap 100 is being applied to a human being. The heat transfer wrap 100 can be applied directly to the surface of the human body for dissipating heat therefrom thereby reducing associated fever and inflammation in soft tissue, surgical incision sites, and problems associated with female menopause, breast feeding/nursing babies, mastitis, and lower back lumbar pain problems.

The heat transfer wrap 100 of the present invention can be preferably provided in the form of a blanket-like article such that the user (not shown) can lay upon or drape over an effected area of the user's body for absorbing heat therefrom. The present invention is ideal for use with toddlers and adults alike and can be utilized to address each of the problems enumerated immediately above. Further, the inventive heat transfer wrap 100 is intended for use at ambient temperature which has been shown during the development and testing phase to be sufficient to lower the body temperature, dissipate heat and reduce inflammation in the body of the user. This significant feature of the present invention enables the heat transfer wrap 100 to be utilized as intended without the requirement of freezing or chilling the wrap 100 prior to use. This advantage distinguishes the present invention over other similar products of the prior art.

In a preferred embodiment, the present invention is embodied in a compartmentalized heat transfer wrap 100 for absorbing heat from a living body (not shown), that includes a moisture proof, generally geometrically-shaped, outer envelope 116 which is sealed about the periphery thereof to form an outer boundary 106 (see FIG. 1). The sealing means is dependent upon the material used to form the outer envelope 106. In the present invention, the prototype design utilizes nylon to form the outer envelope 106 due to nylon being moisture-proof and having heat transfer characteristics. However, other materials can be used such as, for example, robust flexible plastic. Consequently, the sealing of the outer envelope 116 to form the outer boundary 106 can be a pressure heat seal or other suitable sealing means. The material must be of the type that enables the transfer of heat there through and typically is formed in a geometric shape such as a rectangle, square, circle or other desirable shape. The geometric-shaped heat transfer wrap 100 is intended to have a center of gravity 108 (see FIG. 5) that improves the weight distribution and consequently the handling characteristics thereof. In an exemplary embodiment shown in FIGS. 1-6 but in particular FIG. 1, the heat transfer wrap 100 is shown as a rectangular blanket-like article having the dimensions generally in the range of 24″ in length, 17″ in width, and approximately 0.25″ in depth. Another embodiment of the present invention may exhibit the heat transfer wrap 100 in an alternative geometric configuration with different dimensions.

The moisture-proof, outer envelope 116 of the heat transfer wrap 100 is compartmentalized into a plurality of individual sealed quilted pockets 102. Each of the individual sealed pockets 102 is isolated from each of the remaining sealed pockets 102, typically by suitable pressure heat seals 104 or the equivalent (see FIGS. 1-3). During the design and development phase of the present invention, experimentation disclosed that the heat transfer wrap 100 comprising only a single large quilted pocket 102 resulted in a specially formulated cooling medium or gel 120 encapsulated within the single quilted pocket 102 gravitated to the outer boundary 106. This resulted in a dramatic reduction in the heat transfer rate and efficiency and the heat transfer wrap 100 being weighted on the outer boundary 106 and becoming difficult to handle because of uneven weight distribution. Increasing the number of quilted pockets 102 resulted in a greater cooling efficiency and a more even weight distribution since the increased number of quilted pockets 102 prevented the spreading of the cooling gel 120 to the outer boundary 106 by retaining the cooling gel 120 in each respective pocket 102. During experimentation, it has been determined that the eight quilted pockets 102a-102h {as shown in the planar illustration of FIG. 5 of the exemplary embodiment} provides an efficient design having an adequate number of quilted pockets 102 and a suitable weight distribution for an average sized person.

Thus, the number of individual sealed pockets 102 in this preferred embodiment of the heat transfer wrap 100 as illustrated in the accompanying drawing Figs. is eight and are distributed in a rectangular arrangement. This number of sealed pockets 102 has been determined to provide a suitable sized blanket-like article and the conveniently positioned center of gravity 108 to improve handling characteristics. The center point identified by the numeral 108 located at the intersection of diagonals 110 and 112 in FIG. 5 locates the center of gravity of the exemplary embodiment of the heat transfer wrap 100. It is further noted that the compartmentalized heat transfer wrap 100 can be custom designed as desired and that the weight distribution can be manipulated for any specific use. Thus, other arrangements of sealed pockets 102 in other geometric patterns may also be effective. Notwithstanding the geometric shape of the heat transfer wrap 100 and the number of individual quilted pockets 102 formed therein, the outer envelope 116 may be conveniently folded along each of the pressure heat seals 104 for convenient storage.

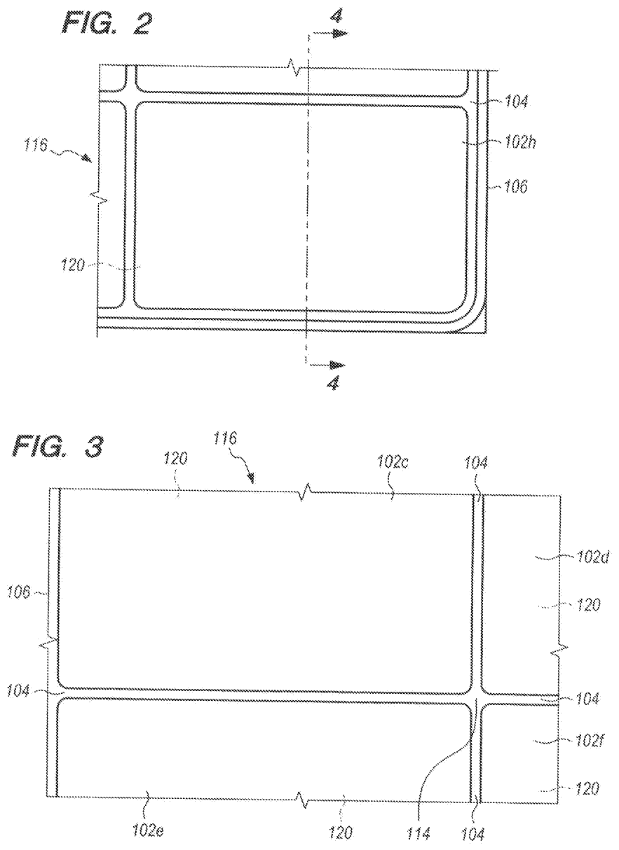

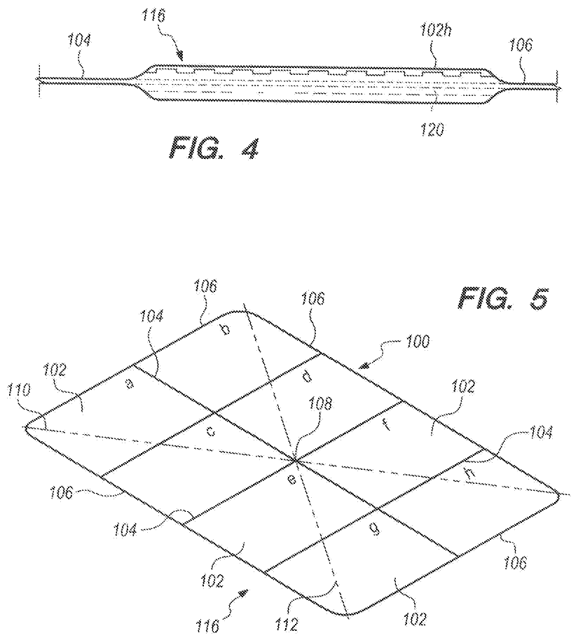

Let us now turn our attention to the specially formulated cooling medium or gel 120 and discuss the components thereof. Each of the individual sealed pockets 102 includes the specially formulated cooling medium or gel 120 encapsulated therein for absorbing heat generated by the living body (not shown) that is in contact with the outer envelope 116 of the heat transfer wrap 100. Thus, the function of the formulated cooling gel 120 is to absorb the heat generated by the living body as illustrated in FIG. 4. A cross-sectional view of the quilted sealed pocket 102h taken along line 4-4 of FIG. 2 is shown in FIG. 4. The quilted sealed pocket 102h shows the outer envelope 116 closed on both ends by the pressure heat seal 104 and the outer boundary seal 106. The formulated cooling gel 120 is shown encapsulated within the outer envelope 116 in FIG. 4. The specially formulated cooling gel 120 includes a combination of components blended to absorb heat including a carboxymethyl cellulose in powder form, water, and a stabilizing additive. This combination of components physically exists as a cooling gel 120 and is designed to functionally absorb heat when employed at ambient temperature, e.g., the cooling gel 120 is not required to be frozen or cooled prior to use. This design feature provides a significant advance over the cooling devices known in the prior art.

The formulation of the cooling gel 120 will now be discussed. The primary components include carboxymethyl cellulose in powder form, water, and a stabilizing additive. The formulated percent of each of the primary components included in a batch of the cooling gel 120 is 4% of carboxymethyl cellulose in powder form, 95.85% water by volume, and 0.15% specific additives as a stabilizing component to maintain freshness. In some cases, alcohol is added to the formulation of the cooling gel 120 for suppressing the growth of mold. The 0.15% of specific additives are comprised of the combination of three ingredients including: {a} 2-bromo-2-nitropropane-1,3-diol (Bronopol); {b} 5-chlor-2-methyl-3(2H)-(isothiazolone); and {c} 2-methyl-3(2H)-(isothiazolone). Once formulated, the consistency of the cooling gel 120 is semi-solid or somewhat fluid. It does not actually flow but pressure must be applied to the quilted sealed pocket 120 to remove the cooling gel 120. The source of the combination of components that form the specially formulated cooling gel 120 is Anhui Komat Ice Packing Company Ltd., 7^{th} North Road, Economic and Technical Area, Luan, Anhui, China. The formulation of the cooling gel 120 is designed to be utilized in the heat transfer wrap 100 while at ambient temperature, e.g., the cooling gel 120 located within the sealed pockets 102 does not require freezing or cooling prior to use. This technical advance in the formulation of the cooling gel 120 in combination with the First Law of Thermodynamics {teaching that heat flows from a warmer body to a cooler body} enables the cooling gel 120 to absorb and then dissipate the heat generated by the living body in contact with the outer envelope 116. For example, a cooling gel 120 at an ambient temperature of 78 degrees Fahrenheit will adequately absorb and lower the temperature in a living body that has a measured temperature of 103 degrees Fahrenheit. The 0.15% of specific additives can be harmful if swallowed and thus care should be exercised when coming in contact with the cooling gel 120.

The heat transfer wrap 100 including the quilted sealed pocket 102 design with the specially formulated cooling gel 120 encapsulated therein has been shown to dramatically increase the heat transfer rate for dissipating the heat from the surface being cooled. Through experimentation, it has also been determined that increasing the number of quilted sealed pockets 102 increases the heat dissipation efficiency of the heat transfer wrap 100. Further, the greater number of quilted pockets 102 provides more manageable weight distribution and improved handling characteristics of the heat transfer wrap 100. This conclusion, of course, reaches the point of diminishing returns as the number of quilted sealed pockets 102 becomes excessive because of {a} manufacturing challenges (pressure heat sealing), and {b} economical cost issues.

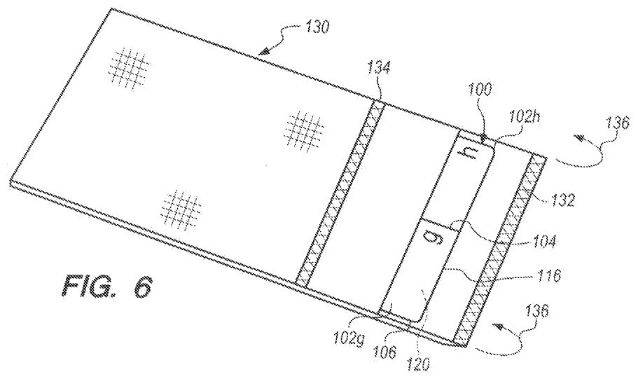

An optional feature of the present invention includes a flat sleeve enclosure 130 for surrounding the outer envelope 116 of the heat transfer wrap 100. The flat sleeve enclosure 130 has dimensions somewhat larger than that of the present invention and surrounds the outer envelope 116 similar to how a mattress cover surrounds a sleeping mattress. Thus, the flat sleeve enclosure 130 will cover the nylon outer envelope 116 so that the outer boundary 106 thereof will fit flatly into the flat sleeve enclosure 130. A closure means such as a hook and loop fastener having a hook strip 132 and a loop strip 134 enables the flat sleeve enclosure 130 to totally enclose the outer envelope 116 by moving the hook strip 132 in the direction shown by numeral 136 in FIG. 6 to mate with the loop strip 134. Opening the flat sleeve enclosure 130 is effected by moving the hook strip 132 in the direction opposite to numeral 136. The outer envelope 116 as presented herein is fashioned from nylon but the flat sleeve envelope 130 can provide a sanitary comfortable surface comprised of a cotton or fleece-like material to change the texture and appearance thereof. Further, the flat sleeve envelope 130 may be laundered regularly when used in conjunction with small children and infants. Further, the flat sleeve envelope 130 can include esthetics, logos, images or designs imprinted thereon, and recognizable tactile characteristics, comfort, and weighted blanket sensory feel, as well as a sanitary guard that are suitable for young children.

An embodiment of the present invention is generally directed to a compartmentalized heat transfer wrap 100 for absorbing heat from a living body including a moisture-proof, geometrically-shaped, outer envelope 116 being sealed about a periphery thereof to form an outer boundary 106. The outer envelope 116 is compartmentalized into a plurality of individual sealed pockets 102 wherein each of the sealed pockets 102 is isolated from each of the remaining individual sealed pockets 102. Further, each of the individual sealed pockets 102 includes a formulated cooling gel 120 encapsulated therein, the cooling gel 120 having a combination of components including carboxymethyl cellulose in powder form, water, and a stabilizing additive for absorbing heat generated by the living body in contact with the outer envelope 116 while the cooling gel 120 is employed and functional at ambient temperature, e.g., the cooling gel 120 does not need to be frozen or cooled prior to use.

Although the construction of the inventive compartmentalized heat transfer wrap 100 is not complicated, it has many practical uses in the domestic environment and advantages over similar prior art devices. For example, it is useful in the following ways: {a} reducing the body temperature of a person suffering with a fever; {b} reducing the effects of menopause in females; {c} reducing inflammation in soft tissue and surgical incisions; {d} direct application to the breasts of nursing mothers suffering from mastitis; and {e} for pain relief and support in the lumbar section of the lower back, to name just a few. Further, each of these uses is accomplished by {f} using the heat transfer wrap 100 with the formulated cooling gel 120 at ambient (room) temperature which is a significant feature of the present invention.

While the present invention is described herein with reference to illustrative embodiments for particular applications, it should be understood that the invention is not limited thereto. Those having ordinary skill in the art and access to the teachings provided herein will recognize additional modifications, applications and embodiments within the scope thereof and additional fields in which the present invention would be of significant utility such as, for example, the medical industry. It is therefore intended by the appended claims to cover any and all such modifications, applications and embodiments within the scope of the present invention. Accordingly,

What is claimed is:

1. A compartmentalized heat transfer wrap for absorbing heat from a living body, said heat transfer wrap comprising:
a moisture proof, outer envelope being sealed about a periphery thereof forming an outer boundary;
said outer envelope being compartmentalized into a plurality of individual sealed pockets wherein each of said sealed pockets is isolated from each of the remaining individual sealed pockets; and
each of said individual sealed pockets includes a formulated cooling medium encapsulated therein, said cooling medium including at least a pair of components identified as carboxymethyl cellulose and water for absorbing heat generated by a living body in contact with said outer envelope while said cooling medium being employed and functional at ambient temperature.

2. The heat transfer wrap of claim 1 wherein said outer boundary is pressure heat sealed.

3. The heat transfer wrap of claim 1 wherein each of said individual pockets is isolated from each of the remaining individual pockets by pressure heat seals.

4. The heat transfer wrap of claim 1 wherein said moisture-proof outer envelope is comprised of nylon.

5. The heat transfer wrap of claim 1 wherein an optimum number of said individual sealed pockets comprises eight pockets.

6. The heat transfer wrap of claim 1 wherein said outer envelope is foldable along each of a plurality of pressure heat seals formed between each of said individual pockets for convenient storage.

7. The heat transfer wrap of claim 1 wherein said cooling medium comprises a cooling gel including at least said carboxymethyl cellulose in powder form and water for absorbing heat while at ambient temperature.

8. The heat transfer wrap of claim 1 wherein said individual sealed pockets of said outer envelope are arranged for including a geometric center of gravity for improved weight distribution and handling.

9. The heat transfer wrap of claim 1 further including a flat sleeve enclosure for surrounding said outer envelope, said flat sleeve enclosure providing a sanitary esthetic, comfortable surface.

10. The heat transfer wrap of claim 9 wherein said flat sleeve enclosure further includes a closure means.

11. The heat transfer wrap of claim 10 wherein said closure means includes a hook and loop closure means.

12. A compartmentalized heat transfer wrap for absorbing heat from a living body, said heat transfer wrap comprising:
a moisture proof, geometrically-shaped, outer envelope being sealed about a periphery thereof forming an outer boundary;
said outer envelope being compartmentalized into a plurality of individual sealed pockets wherein each of said sealed pockets is isolated from each of the remaining individual sealed pockets; and
each of said individual sealed pockets includes a formulated cooling medium encapsulated therein, said cooling medium including a combination of components including carboxymethyl cellulose in powder form, water, and a stabilizing additive for absorbing heat generated by a living body in contact with said outer envelope while said cooling medium being employed and functional at ambient temperature.

13. The heat transfer wrap of claim 12 wherein said stabilizing additive is comprised of the combination of three ingredients including: {a} 2-bromo-2-nitropropane-1,3-diol (Bronopol); {b} 5-chlor-2-methyl-3(2H)-(isothiazolone); and {c} 2-methyl-3(2H)-(isothiazolone).
